(19) United States
(12) Patent Application Publication
(10) Pub. No.: US 2026/0207087 A1
(43) Pub. Date: Jul. 23, 2026

(54) WEARABLE DEVICE FOR CONTINUOUS GLUCOSE MONITORING

(71) Applicant: Allez Health Inc., Carlsbad, CA (US)

(72) Inventor: Robert James Boock, Carlsbad, CA (US)

(73) Assignee: Allez Health Inc., Carlsbad, CA (US)

(21) Appl. No.: 19/448,643

(22) Filed: Jan. 14, 2026

Related U.S. Application Data
(60) Provisional application No. 63/748,707, filed on Jan. 23, 2025.

(51) Int. Cl. A61B 5/145; A61B 5/00; A61B 5/15; A61B 5/155

(52) U.S. Cl. CPC A61B 5/14532; A61B 5/14503; A61B 5/150022; A61B 5/150427; A61B 5/155; A61B 5/6848

(57) ABSTRACT

Disclosed herein is a wearable device for continuous glucose monitoring including a housing having a top surface opposite a bottom surface. A portion of the top surface is moveable relative to the bottom surface. The bottom surface has an opening and is configured to secure directly to skin. A puck assembly has a sensor, a wire coupled to the sensor, and a plurality of contacts. A rigid member is configured to move relative to the bottom surface. An engagement member is configured to rotate about an axis. A cutting element is coupled to the engagement member. Displacement of the movable portion of the top surface moves the rigid member into contact with the engagement member. The cutting element rotates through the opening to form an incision in the skin then to a locked position within the housing. The sensor and the wire advance through the opening and into the incision.

### RELATED APPLICATIONS

This application claims priority to U.S. Provisional Patent Application No. 63/748,707, filed on Jan. 23, 2025, and entitled “Wearable Device for Continuous Glucose Monitoring”; the contents of which are incorporated by reference in full.

### BACKGROUND

Medical patients often have diseases or conditions that require the measurement and reporting of biological conditions. For example, if a patient has diabetes, it is important that the patient have an accurate understanding of the level of glucose in their blood. Traditionally, diabetes patients have monitored their glucose levels by sticking their finger with a small lance, allowing a drop of blood to form, and then dipping a test strip into the blood. The test strip is positioned in a handheld monitor that performs an analysis on the blood and visually reports the measured glucose level to the patient. Based upon this reported level, the patient makes important decisions on what food to consume, or how much insulin to inject into their blood. Although it would be advantageous for the patient to check glucose levels many times throughout the day, many patients fail to adequately monitor their glucose levels due to the pain and inconvenience. As a result, the patient may eat improperly or inject either too much or too little insulin. Either way, the patient has a reduced quality of life and increased chance of doing permanent damage to their health and body.

Diabetes is a devastating disease that if not properly controlled can lead to terrible physiological conditions such as kidney failure, skin ulcers, or bleeding in the eyes, and eventually blindness, pain and the eventual amputation of limbs.

Regular and accurate monitoring of glucose levels is critical for diabetes patients. To facilitate such monitoring, continuous glucose monitoring (CGM) sensors are a type of device in which glucose is automatically measured from fluid sampled in an area just under the skin multiple times a day. CGM devices typically involve a small housing in which the electronics are located and which is adhered to the patient's skin to be worn for a period of time. A small needle within the device delivers the subcutaneous sensor which is often electrochemical. In this way, a patient may install a CGM on their body, and the CGM will provide automated and accurate glucose monitoring for many days without any action required from the patient or a caregiver. It will be understood that depending upon the patient's needs, that continuous glucose monitoring may be performed at different intervals. For example, some continuous glucose monitors may be set or programmed to take multiple readings per minute, whereas in other cases the continuous glucose monitor can be programmed or set to take readings every hour or so. It will be understood that a continuous glucose monitor may sense and report readings at different intervals.

Continuous glucose monitoring is a complicated process, and it is known that glucose levels in the blood can significantly rise/increase or lower/decrease quickly, due to several causes. A single glucose measurement provides only a snapshot of the instantaneous level of glucose in a patient's body. Such a single measurement provides little information about how the patient's use of glucose is changing over time, or how the patient reacts to specific dosages of insulin. Accordingly, even a patient that is adhering to a strict schedule of strip testing will likely be making incorrect decisions as to diet, exercise, and insulin injection. Of course, this is exacerbated by a patient that is less consistent on performing their strip testing. To give the patient a more complete understanding of their diabetic condition and to get a better therapeutic result, some diabetic patients use continuous glucose monitoring.

Electrochemical glucose sensors operate by using electrodes which typically detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is biased. The reference electrodes essentially complete the electron flow in the electrochemical circuit. Three-electrode (or three-pole) designs have a working electrode, a reference electrode and a counter electrode. The counter electrode replenishes ionic loss at the reference electrode and is part of an ionic circuit.

Conventional CGM systems typically use a working wire that uses a core of tantalum on which a thin layer of platinum is deposited. Tantalum is a relatively stiff material, so is able to be pressed into the skin without bending, although an introducer needle may be used to facilitate insertion. Further, it is inexpensive as compared to platinum, which makes for an economical working wire. As is well known, an enzyme layer is deposited over the platinum layer, which is able to accept oxygen molecules and glucose molecules from the user's blood. The key chemical processes for glucose detection occur within the enzyme membrane. Typically, the enzyme membrane has one or more glucose oxidase enzymes (GOx) dispersed within the enzyme membrane. When a molecule of glucose and a molecule of oxygen (O₂) are combined in the presence of the glucose oxidase, a molecule of gluconate and a molecule of hydrogen peroxide (H₂O₂) are formed. In one construction, the platinum surface facilitates a reaction wherein the hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which is intended to be related to the number of glucose molecules oxidized. A measurement of the electrical current on the platinum wire can thereby be associated with a particular level of glucose in the patient's blood or interstitial fluid (ISF).

Unfortunately, the cost of using a continuous glucose monitor may be prohibitive for many patients that could benefit greatly from its use. As described generally above, a continuous glucose monitor has two main components. First, there is a housing for the electronics, processor, memory, wireless communication, and power. The housing is typically reusable over extended periods of time, such as months. This housing then connects or communicates to a disposable CGM sensor that is adhered to the patient's body, which typically uses an introducer needle to subcutaneously insert the sensor into the patient. This sensor must be replaced, sometimes as often as every three days, and likely at least once every other week. Thus, the cost to purchase new disposable sensors represents a significant financial burden to patients and insurance companies. Because of this, a substantial number of patients that could benefit from continuous glucose monitoring are not able to use such systems and are forced to rely on the less reliable and painful finger stick monitoring.

The working wire is then associated with a reference electrode, and in some cases one or more counter electrodes, which form the CGM sensor. In operation, the CGM sensor is coupled to and cooperates with electronics in a small housing in which, for example, a processor, memory, a wireless radio, and a power supply are located. The CGM sensor typically has a disposable applicator device that uses a small introducer needle to deliver the CGM sensor subcutaneously into the patient. Once the CGM sensor is in place, the applicator is discarded, and the electronics housing is attached to the sensor. Although the electronics housing is reusable and may be used for extended periods, the CGM sensor and applicator need to be replaced quite often, usually every few days. In such known CGM sensors, the electronics housing has all the supporting electronics for the sensor in the sensor housing, such as an analog front end, processor, memory, and radio, as well as the battery. Typically, the battery will have some trickle-power sensing circuit that can detect when the electronics housing is coupled to the CGM sensor. Once such a detection is sensed, then the battery can be used to fully power the electronics and the working wire in the CGM sensor. In this way, the battery must be sized to allow for low-power sensing for extended periods of time, which can extend for a year or more, and have sufficient reserve power to operate the CGM sensors that it detects. As the electronics housing is reusable on multiple CGM sensors, the battery must be sized to handle the expected number of uses.

Despite advancements in CGM applicator technology, challenges remain in simplifying the insertion process, enhancing user comfort, and ensuring safe disposal, highlighting the need for a self-inserting sensor.

### SUMMARY

Disclosed herein is a wearable device for continuous glucose monitoring including a housing having a top surface opposite a bottom surface. A portion of the top surface is moveable relative to the bottom surface. The bottom surface has an opening and is configured to secure directly to skin of a patient. A puck assembly is disposed within the housing. The puck assembly has a sensor, a wire coupled to the sensor, and a plurality of contacts. A rigid member is disposed within the housing and configured to move relative to the bottom surface. An engagement member is disposed within the housing, spaced apart from the rigid member in an initial position and configured to rotate about an axis. A cutting element is coupled to the engagement member. Displacement of the movable portion of the top surface moves the rigid member into contact with the engagement member and produces rotation of the engagement member and the cutting element about the axis. The cutting element rotates through the opening to form an incision in the skin then to a locked position within the housing. The locked position secures the cutting element away from the skin. The sensor and the wire advance through the opening and into the incision when the movable portion of the top surface is displaced.

Disclosed herein is a method for implanting a sensor using a wearable device for continuous glucose monitoring. The method includes positioning a wearable device in contact with skin of a patient. The wearable device includes a housing having a top surface and a bottom surface. The bottom surface includes an opening. A sensor and wire are disposed within the housing. An engagement member and a rigid member are disposed within the housing. A cutting element is coupled to the engagement member. The bottom surface of the housing contacts the skin of the patient. A force is applied to the top surface to move a portion of the top surface relative to the bottom surface. The rigid member contacts the engagement member, and the engagement member and the cutting element rotates about an axis. The cutting element rotates through the opening to form an incision in the skin. The cutting element retracts out of the opening to a locked position within the housing securing the cutting element away from the skin. The sensor and wire are directed through the opening and into the incision.

### BRIEF DESCRIPTION OF THE DRAWINGS

FIG. 1A is a perspective view of a prior art applicator for a glucose monitoring system.

FIG. 1B is a perspective view of a prior art applicator and prior art wearable device for a glucose monitoring system.

FIG. 2A is a perspective view of a wearable device with a self-inserting sensor, in accordance with some aspects.

FIG. 2B is a side view of the wearable device with self-inserting sensor, in accordance with some aspects.

FIG. 3 depicts a cross-sectional view A-A of FIG. 2A, in accordance with some aspects.

FIGS. 4A-4C show close-up views of components within the housing, all in accordance with some aspects.

FIGS. 5A and 5B illustrate cross-sectional views of the wearable device after actuation of the top surface, both in accordance with some aspects.

FIG. 6 illustrates example profiles of the cutting element, in accordance with some aspects.

FIG. 7A shows a wearable device with a self-inserting sensor, in accordance with some aspects.

FIG. 7B depicts a cross-sectional view B-B of FIG. 7A, in accordance with some aspects.

FIG. 7C is a cross-sectional view B-B of FIG. 7A, in accordance with some aspects.

FIG. 8 is a flowchart for a method for implanting a sensor using a wearable device for continuous glucose monitoring, in accordance with some aspects.

### DETAILED DESCRIPTION

Continuous glucose monitoring (CGM) systems require the insertion of a sensor into the subcutaneous tissue of a user. The sensor is typically inserted using a dedicated applicator device designed to ensure proper placement of the sensor while minimizing user discomfort. These applicators may be preloaded with the sensor, or the user may need to attach the sensor to the applicator before insertion. In either case, the applicators known in the art are configured for single use to maintain sterility and reduce the risk of infection.

Conventionally, CGM applicators are primarily activated through manual or mechanical means. Many applicators utilize a spring-loaded mechanism, triggered by pressing a button or other user action, to drive a sharp needle carrying the sensor into the tissue of the user. Once the sensor is positioned, the needle is retracted or disengaged from the sensor, leaving the sensor in place and securely embedded in the tissue. Some applicators employ features like safety locks or shields to prevent accidental activation during handling.

After use, the applicators and the needle are typically discarded as single-use devices. This approach ensures sterility during sensor insertion and eliminates the need for cleaning or reprocessing. Some applicators incorporate mechanisms to retract or shield the needle after activation to enhance safety and reduce the risk of accidental needle sticks during disposal.

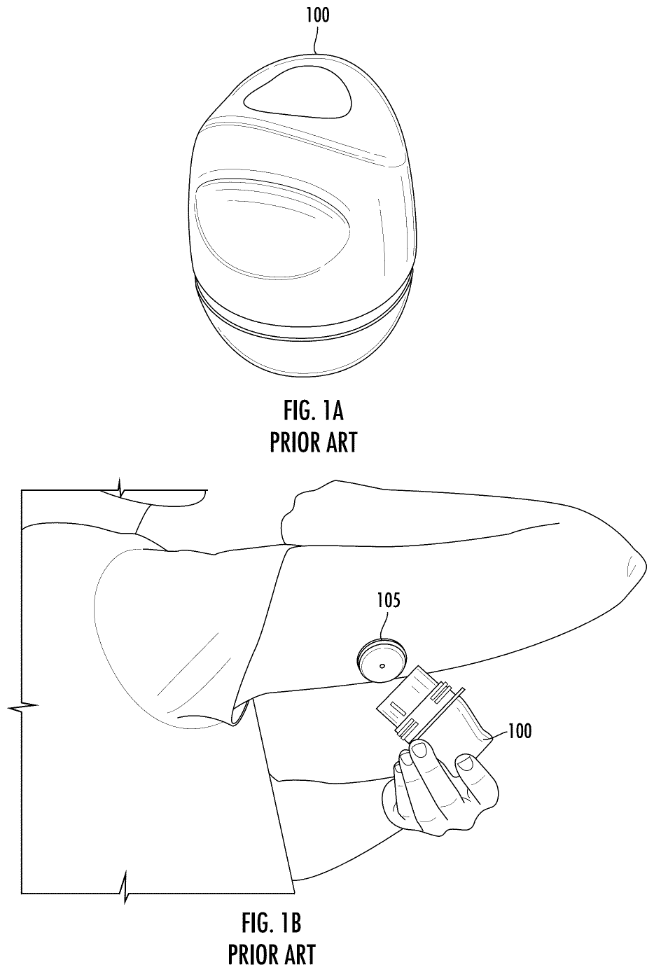

FIG. 1A is a perspective view of a prior art applicator for a glucose monitoring system, and FIG. 1B is a perspective view of a prior art applicator and prior art wearable device for a glucose monitoring system, both as known in the art. The applicator 100 is used to place the sensor subcutaneously into the patient, leaving wearable device 105 generally placed over the sensor site. In some cases, the sensor is pre-coupled to the wearable device 105, allowing the combined unit to be positioned on the patient's skin and secured using an adhesive or other attachment mechanism. In this configuration, the sensor is inserted into the subcutaneous tissue by the applicator 100. In other cases, the sensor is initially coupled to the applicator 100 that facilitates accurate insertion into the patient. Once inserted, the applicator 100 is removed, and the wearable device 105 is subsequently attached to the sensor, securing it for continuous glucose monitoring.

Generally, the applicator 100 requires a vertical insertion method for implanting the sensor underneath the skin. To use the applicator 100, the bottom surface of the applicator 100 is placed on the skin of the patient, and a downward force is applied on the top surface of the applicator 100 deploying a needle with the sensor for placing the sensor underneath the skin. After the sensor is implanted, the needle automatically retracts and the wearable device 105 only—meaning without the applicator—is adhered to the skin. The applicator 100 (including any coupled components) are disposable. This is typically a two-component system, consisting of a wearable device and a separate applicator. The wearable device is designed to be worn on the skin, while the applicator is a disposable component intended for single use.

As described, conventional CGM systems rely on external applicators for sensor insertion, which add complexity, cost, and waste to the process. These applicators often require precise manufacturing to ensure sterility, accuracy, and safety during use, and their single-use nature generates additional medical waste. Furthermore, applicators can be intimidating or challenging for some users, particularly those with limited dexterity, visual impairments, or a fear of needles.

Systems and methods herein disclose a wearable device with a self-inserting sensor for continuous glucose monitoring which may be part of a continuous glucose monitoring (CGM) system. A self-inserting sensor refers to a sensor capable of independently inserting itself into the subcutaneous tissue of a user without the need for a separate applicator device. Such a device may combine both the sensor and insertion mechanism in a single unit, simplifying the overall process for users. This simplifies the process of sensor insertion by eliminating the need for a separate applicator. This integrated design reduces the number of components required, streamlining the manufacturing and assembly process while minimizing user handling steps. The combination of a sensor and an insertion mechanism in a single device enhances ease of use, particularly for individuals with limited dexterity or those unfamiliar with CGM systems, thereby promoting greater accessibility and adoption of the technology.

Unlike conventional applicators that rely on a sharp introducer needle to create an insertion path, the wearable devices disclosed herein form the incision using a rotating cutting element integrated within the housing. This configuration eliminates the need for a needle, significantly reducing user anxiety and discomfort commonly associated with needle-based systems. The design ensures precise and controlled placement of the sensor beneath the skin without piercing by a needle. The cutting element creates or forms a minimal incision and immediately retracts into a locked position within the housing after use, providing a safe, needle-free experience that enhances user confidence and comfort.

The self-inserting configuration ensures precise alignment and placement of the sensor within the subcutaneous tissue, reducing the potential for user error during installation. This design also enhances portability by combining functionality into a compact form factor, making it easier for users to carry and deploy the system in various settings. By consolidating sensor insertion into the wearable device, the overall system is made more intuitive and efficient, leading to a seamless user experience and improving consistency in sensor performance during operation. This improves the user experience by reducing the number of components, lowering costs, and minimizing the environmental impact.

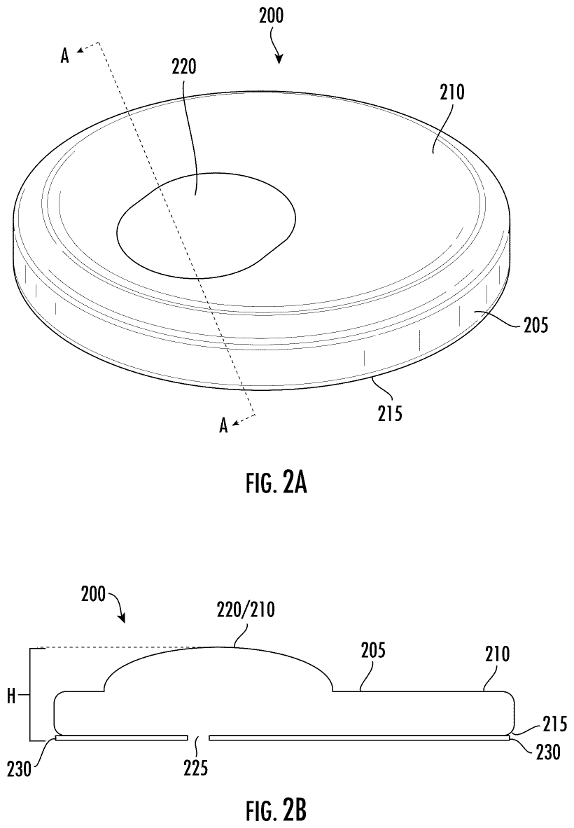

FIG. 2A is a perspective view of a wearable device with a self-inserting sensor, and FIG. 2B is a side view of the wearable device with self-inserting sensor, both in accordance with some aspects. A wearable device 200 with a self-inserting sensor may be of any shape such as a circular, oval, rectangular, square, or other geometries that provide a compact and comfortable form factor for the user. The wearable device 200 may have a flat, low-profile design to minimize intrusion during daily activities. In some aspects, the wearable device 200 has a height H above the skin. The height H may be 5.5 mm to 9 mm. The design allows the wearable device 200 to adhere securely to the skin, with a thin and lightweight construction to reduce bulk. This configuration enables the wearable device 200 to be worn for extended periods without restricting movement or causing discomfort.

The wearable device 200 includes a housing 205 having a top surface 210 positioned opposite a bottom surface 215. At least a portion of the top surface 210, or a component in/on/of the top surface, is configured to move relative to the bottom surface 215. In some implementations, the top surface 210 incorporates a raised section forming a dome shaped structure, referred to as dome 220 in this example. This dome portion may serve as the movable section of the top surface 210, enabling actuation with respect to the bottom surface 215. The bottom surface 215 is configured to directly contact skin of a patient and may include an opening 225 in the bottom surface 215. An adhesive pad 230 is coupled to the bottom surface 215, surrounds the opening 225, and is configured to adhere the wearable device 200 to the user's skin. The housing 205, including the dome 220, may be comprised of a flexible material such as a medical-grade silicone, thermoplastic elastomer (TPE), polyurethane, and/or similar biocompatible polymers to provide comfort and durability. In some examples, multiple materials are used, such as a rigid material for the main housing combined with a flexible material for the dome.

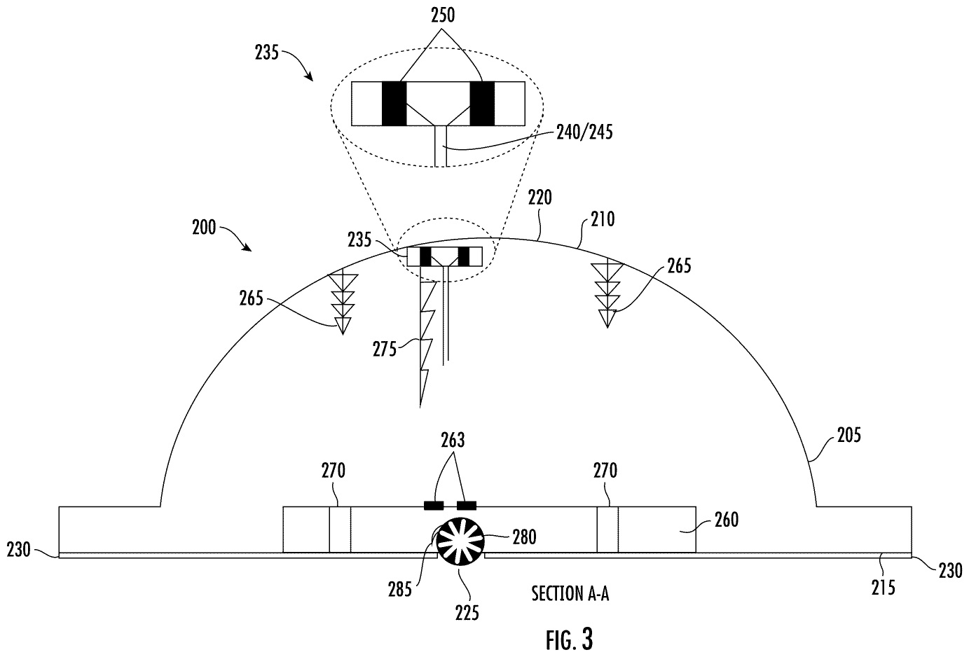

FIG. 3 depicts a cross-sectional view A-A of FIG. 2A, in accordance with some aspects. A puck assembly 235 is disposed within the housing 205. In this example, the puck assembly 235 is coupled to the top surface 210 of the housing 205 in the dome 220. The puck assembly 235 includes a sensor 240, a wire 245 and a plurality of contacts 250. The sensor 240 is configured to generate one or more signals associated with an in vivo glucose level in a bodily fluid under the skin of the patient. A circuit board 260 may be disposed within the housing 205 and, in this example, coupled to the bottom surface 215 of the housing 205. Terminals 263 on the circuit board 260 are configured to electrically couple to the plurality of contacts 250 of the puck assembly 235.

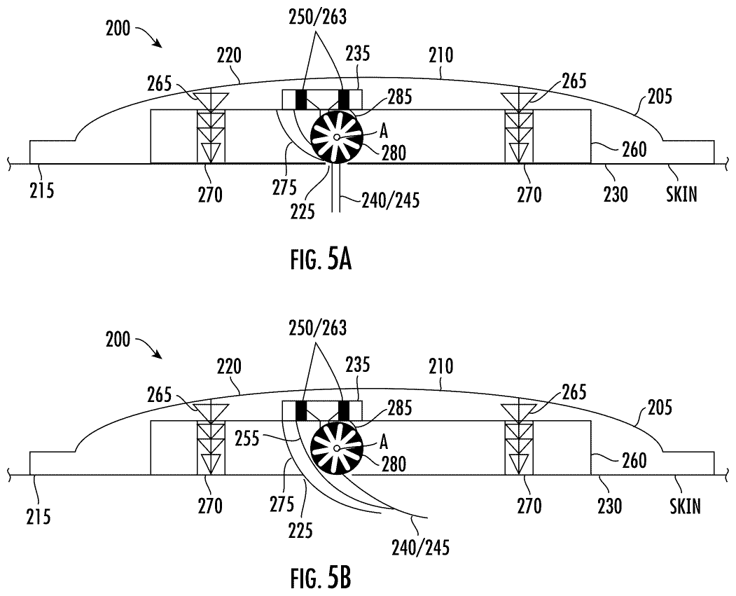

In some aspects, a stepped shaft 265 extends from the top surface 210 of the housing 205 toward the bottom surface 215, and the circuit board 260 includes a complementary recess 270. In some implementations, two stepped shafts may be provided; however, the design may include a single stepped shaft or more than two shafts depending on structural requirements. The stepped shaft 265 is received within the recess 270 when the top surface is actuated as shown in FIGS. 5A and 5B. The stepped shaft 265, when engaged with the recess 270, secures the top surface 210 in a collapsed, locked position.

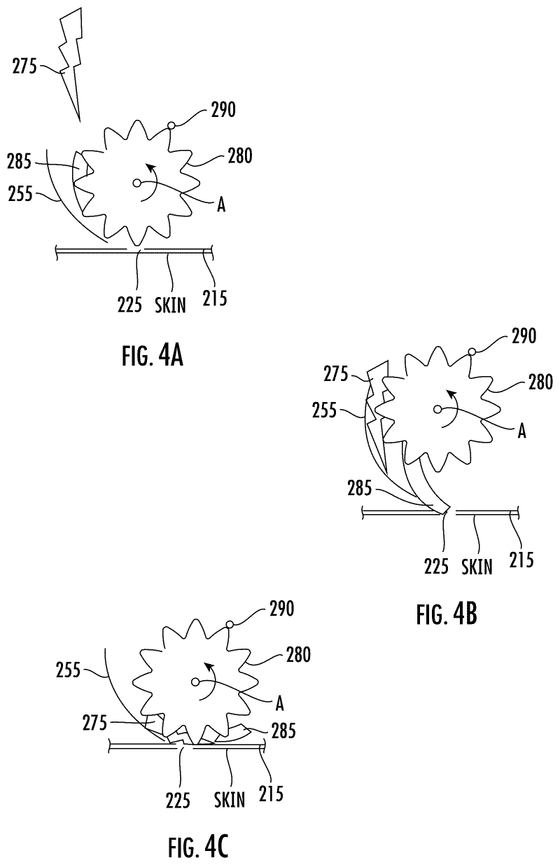

FIGS. 4A-4C show close-up views of components within the housing, all in accordance with some aspects. Referring to FIGS. 3 and 4A-4C, a rigid member 275 is disposed within the housing 205 and configured to move relative to the bottom surface 215. In FIGS. 4A-4C, the adhesive pad 230 is not shown for clarity. An engagement member 280 is disposed within the housing 205 and spaced apart from the rigid member 275 in an initial position also referred to as an undeployed position (shown in FIG. 4A). The engagement member 280 is configured to rotate about an axis A and configured to engage with the rigid member 275 during actuation (shown in FIG. 4B). A cutting element 285 is coupled to the engagement member 280 and rotates about the axis A together with the engagement member 280. A guide 255 is disposed within the housing 205 and configured to direct the sensor 240 and the associated wire 245 (not shown in FIGS. 4A-4C) toward the opening 225 of the bottom surface 215. The guide 255 may be positioned adjacent to or in proximity with the cutting element 285, and in some implementations, the guide 255 may be attached to the cutting element 285. In some aspects, the guide 255 may be implemented as a hollow tube, sleeve, or channel.

FIG. 4A shows an initial position of nonengagement between the rigid member 275 and engagement member 280 which are spaced apart. In this position, the cutting element 285 coupled to the engagement member 280 is positioned away from the bottom surface 215. In some examples, the engagement member 280 is circular, and the cutting element 285 is mounted on an upper surface or along the circumferential side of the engagement member 280, positioned away from the bottom surface 215.

FIG. 4B illustrates an engaged position in which the rigid member 275 contacts the engagement member 280 and initiates rotation of the engagement member about axis A. To implant the sensor 240, a force is applied by the user to the top surface 210 of the wearable device 200, and the rigid member 275 advances toward and contacts or engages with the engagement member 280. This engagement imparts a rotation (counterclockwise in this example) to the engagement member 280 relative to its initial position shown in FIG. 4A. As the engagement member 280 rotates, the cutting element 285 coupled thereto moves toward the opening 225 in the bottom surface 215 and advances through the opening 225 to form a precise incision, such as a cut or nick, in the patient's skin. Following the incision, the cutting element 285 continues its rotational path, retracting into the housing 205 and away from the skin.

FIG. 4C illustrates the engagement member 280 in a locked configuration. After the cutting element 285 retracts into the housing 205 and away from the skin, the engagement member 280 is immobilized to prevent further rotation. A stop mechanism 290 disposed within the housing 205 secures the engagement member 280 in the locked or fixed position and prevents reverse rotation or additional movement. The stop mechanism 290 may be a mechanical latch, detent, notch or other locking feature configured to maintain the engagement member 280 in the locked state. In the locked state, the cutting element 285 rests on an upper surface or along the circumferential side of the engagement member 280, positioned safely away from the opening 225 in the bottom surface 215 (e.g., the patient's skin) and rendered immobile. The cutting element 285 remains securely stored within the housing 205 for the entire duration of sensor wear, eliminating the risk of accidental contact with the skin.

Displacement of the movable portion of the top surface 210 moves the rigid member 275 into contact with the engagement member 280 and produces rotation of the engagement member 280 and the cutting element 285 about the axis A. The cutting element 285 rotates through the opening 225 to form an incision in the skin and then to a locked position within the housing 205. The locked position secures the cutting element 285 away from the skin. The sensor 240 and the wire 245 advance through the opening 225 and into the incision when the movable portion of the top surface 210 is displaced.

In some examples, the engagement member 280 comprises a gear, and the rigid member 275 comprises as a rod. The gear includes teeth configured to mesh with corresponding features on the rod during rotation. In some implementations, the rod may also have stepped or toothed surface to enhance engagement such as a rack (275) and pinion (280). When the rod (e.g., rigid member 275) engages the gear (e.g., engagement member 280), rotational movement is imparted to the gear driving the cutting element 285 toward and through the opening 225 for incision. This configuration can further function as a stop mechanism 290 by preventing reverse movement once engaged, similar to a tie strap or ratchet system, ensuring that the engagement member 280 remains locked in position after actuation and cannot be backed out. The stop mechanism 290 may include a pawl configured to engage the engagement member 280 (ratchet) and prevent rotation in the reverse direction.

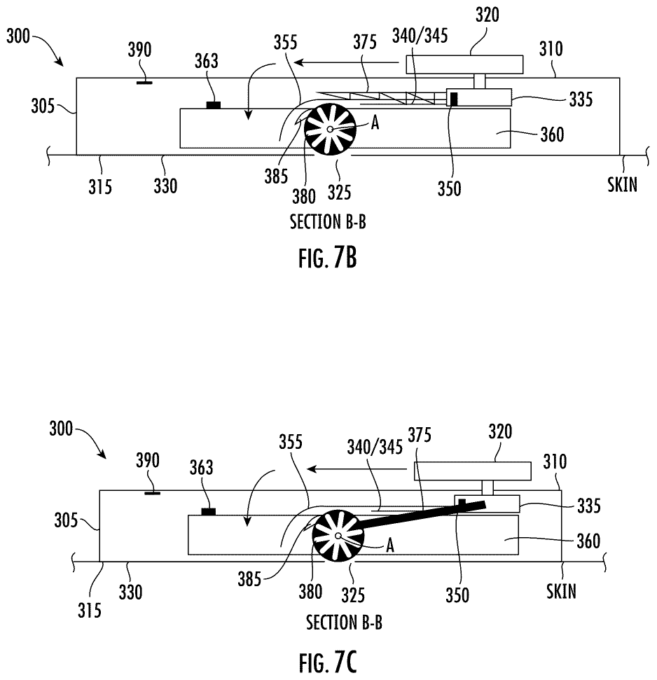

In other examples, the engagement member 280 comprises a disk, and the rigid member 275 comprises a plunger. Similar to the gear-and-rod configuration, engagement between the plunger (e.g., rigid member 275) and the disk (e.g., engagement member 280), transfers rotational movement to the disk to move the cutting element 285. This is shown in FIG. 7C.

FIGS. 5A and 5B illustrate cross-sectional views of the wearable device 200 after actuation of the top surface 210, both in accordance with some aspects. When a force is applied to the top surface 210, a portion of the top surface 210 (e.g. the dome 220) moves relative to the bottom surface 215. In this example, the portion of the top surface 210 is configured to move perpendicular to and toward the bottom surface 215. As the portion of the top surface 210 moves, the puck assembly 235 advances toward the circuit board 260, and the plurality of contacts 250 on the puck assembly 235 electrically couple to corresponding terminals 263 on the circuit board 260. Concurrently, the sensor 240 and associated wires 245 are guided through the opening 225 and into the incision created in the patient's skin, thereby implanting the sensor. During actuation, the rigid member 275 may rotate beneath and slightly around the engagement member 280 within the housing 205. After actuation, the rigid member 275 remains in this “tucked” positioned for the duration of sensor wear.

FIG. 5B depicts a guide 255 disposed within the housing 205 and aligned with the opening 225. The guide 255 is configured to direct the sensor 240 and wire 245 toward the opening 225 during insertion. In some aspects, the guide 255 may form a tubular sleeve surrounding the wire 245 to provide structural reinforcement, preventing column buckling and ensuring precise alignment of the sensor 240. In certain implementations, the guide 255 may extend through the opening 225 and into the incision, remaining positioned within the skin for the duration of sensor wear.

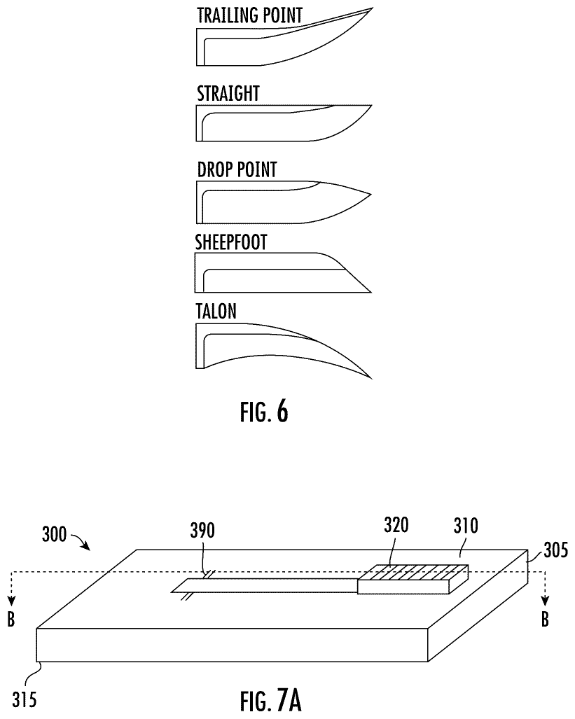

FIG. 6 illustrates example profiles of the cutting element, in accordance with some aspects. The cutting element 285 is coupled to the engagement member 280 and includes a blade configured to form an incision in the skin. The blade may have a profile selected from trailing-point, straight, drop-point, sheepsfoot, talon, or other similar configurations.

FIG. 7A shows a wearable device with a self-inserting sensor, and FIG. 7B depicts a cross-sectional view B-B of FIG. 7A, both in accordance with some aspects. In this example, a wearable device 300 with a self-inserting sensor is rectangular in shape having a housing 305 with a top surface 310 and a bottom surface 315. A portion of the top surface 310 may be moveable relative to the bottom surface 315 (e.g., moveable portion of the top surface 310). In some implementations, the movable portion of the top surface 310 includes a raised section forming a slider 320, as illustrated in this example. The slider 320 functions as the movable portion of the top surface 310 and enables actuation with respect to the bottom surface 315. As shown in FIGS. 2B and 3, the bottom surface 315 is configured to contact the patient's skin and includes an opening 325. An adhesive pad 330 is coupled to the bottom surface 315, surrounding the opening 325, meaning the adhesive does not cover the opening 325, and is configured to attach the wearable device 300 securely to the patient's skin.

Inside of the housing 305, a puck assembly 335 is disposed within the housing 305. In this example, the puck assembly 335 may be coupled to the top surface 310 of the housing 305 such as the slider 320. The puck assembly 335 includes a sensor 340, a wire 345 and a plurality of contacts 350. The sensor 340 is configured to generate one or more signals associated with an in vivo glucose level in a bodily fluid under the skin of the patient. A circuit board 360 may be disposed within the housing 305 and, in this example, coupled to the top surface 310 of the housing 305. Terminals 363 on the circuit board 360 are configured to electrically couple to the plurality of contacts 350 of the puck assembly 335.

A rigid member 375 is positioned within the housing 305 and configured to move relative to the bottom surface 315. An engagement member 380 is spaced apart from the rigid member 375 in its initial state. The engagement member 380, illustrated as a gear with teeth, is configured to rotate about axis A and configured to interact with the rigid member 375, which may include teeth or stepped features to mesh with the gear. A cutting element 385 is coupled to the engagement member 380 for performing the incision. A guide 355 is also disposed within the housing 305 and aligned to direct the sensor 340 and its associated wire 345 toward the opening 325 in the bottom surface 315. The guide facilitates a directional change of the sensor 340 and wire 345 from movement parallel to the bottom surface 315 to movement perpendicular to the bottom surface 315, ensuring accurate placement and structural support during insertion.

When a force is applied to the top surface 310, a portion of the top surface 310 (e.g., slider 320) moves relative to the bottom surface 315. In other aspects, the movable portion may be referred to as a component of the top surface or a separate actuation element integrated into the housing. The movable portion may include a raised section, a recessed section, or a distinct actuator pad configured to translate or pivot relative to the bottom surface. In this example, the top surface 310 is configured to move laterally with respect to the bottom surface 315. As the portion of the top surface 310 moves, the puck assembly 335 advances laterally, and the plurality of contacts 350 on the puck assembly 335 electrically couple to corresponding terminals 363 on the circuit board 360.

Movement of the movable portion of the top surface 310 relative to the bottom surface 315 enables contacts between the rigid member 375 and the engagement member 380. This interaction imparts rotation to the engagement member 380 from its initial position. As the engagement member 380 rotates, the cutting element 385 coupled thereto advances toward the opening 325 in the bottom surface 315, passes through the opening 325, and creates a precise incision in the patient's skin. Following the incision, the cutting element 385 continues its rotational path, retracting fully into the housing 305 and away from the skin for safe storage. Simultaneously, the sensor 340 and associated wire 345 are guided through the opening 325 by the guide 355 and positioned into the incision, thereby implanting the sensor securely beneath the skin.

A stop mechanism 390 locks the engagement member 380 in a fixed position, preventing any further movement. In this example, the stop mechanism 390 is implemented as a groove or notch located at the end of the travel path of the slider 320. When the slider 320 reaches this position, it engages the groove or notch, securing the mechanism. In the locked state, the rigid member 375 remains in contact with the engagement member 380 and cannot retract or disengage, ensuring the cutting element remains safely stored within the housing for the duration of use.

FIG. 7C is a cross-sectional view B-B of FIG. 7A, in accordance with some aspects. In this example, the rigid member 375 may be implemented as a piston, and the engagement member 380 may be configured as a disk rotatable about axis A. The engagement member 380 is designed to interact with the rigid member 375 by receiving a linear force from the piston and converting it into rotational motion about axis A. This rotational movement drives the cutting element 385 coupled to the engagement member 380 toward the opening 325 in the bottom surface 315 for incision, followed by retraction into the housing 305 for safe storage.

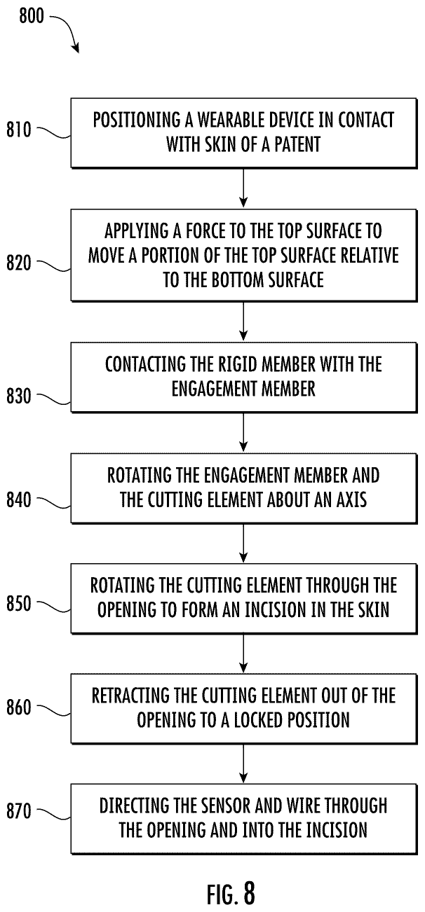

FIG. 8 is a flowchart for a method for implanting a sensor using a wearable device for continuous glucose monitoring, in accordance with some aspects. The particular steps, order of steps, and combination of steps are shown for illustrative and explanatory purposes only. Other examples can implement different particular steps, orders of steps, and combinations of steps to achieve similar functions or results. A method 800 for implanting a sensor using a wearable device for continuous glucose monitoring begins at block 810 by positioning a wearable device in contact with skin of a patient. The wearable device includes a housing having a top surface and a bottom surface. The bottom surface includes an opening, and the bottom surface of the housing contacts the skin of the patient. A sensor and wire are disposed within the housing. An engagement member and a rigid member are also disposed within the housing. A cutting element is coupled to the engagement member. It will be appreciated that the components and operation described herein can be applied to this example.

At block 820, a force is applied to the top surface to move a portion of the top surface relative to the bottom surface. The force may be applied perpendicular to the bottom surface of the housing or laterally relative to the bottom surface. At block 830, the rigid member contacts the engagement member. At block 840, the engagement member and the cutting element rotate about an axis. At block 850, the cutting element rotates through the opening of the bottom surface to form an incision in the skin. At block 860, the engagement member continues rotation, and the cutting element retracts out of the opening to a locked position within the housing securing the cutting element away from the skin. At block 870, the sensor and wire are directed through the opening and into the incision. The method 800 implants the sensor without a separate applicator.

The following example describes the insertion process for wearable device 200, but the same steps apply to wearable device 300. To implant the self-inserting sensor 240 of wearable device 200 into a patient's skin, the bottom surface 215 with the adhesive pad 230 is positioned on the patient's skin. A force is then applied to the top surface 210 of the housing 205. In some aspects, the force is applied perpendicular to the bottom surface 215, causing a portion of the top surface 210 (e.g., dome 220 of wearable device 200) to move toward the bottom surface 215. In other aspects, the force is applied laterally relative to the bottom surface 315, causing a portion of the top surface 310 (e.g., slider 320 of wearable device 300) to move toward the bottom surface 315.

During actuation, the rigid member 275 moves toward and engages with the engagement member 280, rotating the engagement member 280. The cutting element 285 coupled to the engagement member 280 also rotates and advances through the opening 225 in the bottom surface 215 of the housing 205 and into the skin of the patient, forming an incision. Subsequently, the cutting element 285 retracts out of the skin as the engagement member 280 continues rotating. After the cutting element 285 retracts out of the skin, the stop mechanism 290 secures the engagement member 280 in a fixed position preventing movement.

As shown in FIG. 4A, the rigid member 275 and the engagement member 280 are in an initial non-engaged position, where the rigid member 275 and the engagement member 280 are not interacting, and the cutting element 285 is positioned away from the skin. From this state, the rigid member 275 and the engagement member 280 transition to an engaged position, where interaction between the rigid member 275 and engagement member 280 causes the cutting element 285 to rotate through the bottom surface 215 of the housing 205 and into the skin. Then, the rigid member 275 and engagement member 280 move to a final locked position, where a stop mechanism 290 activates to secure the engagement member 280 in place, preventing further movement. In this locked position, the rigid member 275 and the engagement member 280 may remain engaged, but the cutting element 285 rotates out of the skin and away from the bottom surface 215 of the housing 205.

A guide 255 directs the sensor 240 and wire 245 into the incision, and the circuit board 260 receives the plurality of contacts 250. Put another way, the act of pushing down the dome 220 of the top surface 210 causes the engagement member 280 to rotate so that the cutting element 285 pierces the skin. The guide 255 directs the sensor 240 and the wire 245 into the incision and implants the sensor 240 and the wire 245. In some aspects, the wire 245 and sensor 240 may be positioned within the guide 255 which may be located adjacent to the engagement member 280. The guide 255 is configured to support the wire 245, ensuring structural stability and preventing column buckling when force is applied, such as by pressing on the top surface 210. Additionally, the guide 255 provides slight resistance to the wire 245, enabling controlled movement during operation. This configuration directs the wire 245 smoothly, minimizes deflection, and maintains precise alignment throughout the process, facilitating reliable and accurate performance.

The cutting element 285 is very precise and may be similar to the tip of a razor blade or a curved design such as shaped as a beak with the cutting element 285 on the concave surface. The cutting element 285 forms a minimal incision by breaking the skin without significant penetration. In some aspects, the sensor 240 is implanted 4 mm to 6 mm beneath the skin. Since the cutting element 285 rotates into and then out of the skin, it is not left within the incision. Additionally, the cutting element 285 remains securely housed within the housing 205 of the wearable device 200 in a locked position throughout the use of the wearable device 200.

The self-insertion of the sensor 240 operates by rotating and cutting, creating an incision, immediately followed by the sensor 240 being guided into the incision. The cutting and inserting of sensor 240 occur almost simultaneously, minimizing any time lapse between the creation of the cut and the wire/sensor entry into the opening. This immediate insertion is critical, as delayed insertion allows the skin to begin closing, increasing resistance and making the process more difficult. Additionally, prompt insertion ensures that the wire 245 is properly supported, preventing column buckling, which can occur if the wire 245 is left unsupported or if excessive resistance is encountered during the delay.

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific aspects of the invention, it will be appreciated that those skilled in the art, upon attaining an understanding of the foregoing, may readily conceive of alterations to, variations of, and equivalents to these examples. For instance, features illustrated or described as part of one example may be used with another example to yield a still further example. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

1.-7. (canceled)

8. A wearable device for continuous glucose monitoring, comprising:
a housing having a top surface opposite a bottom surface, a portion of the top surface moveable relative to the bottom surface, the bottom surface having an opening and configured to secure directly to skin of a patient;
a puck assembly disposed within the housing, the puck assembly having a sensor, a wire coupled to the sensor, and a plurality of contacts;
a rigid member disposed within the housing and configured to move relative to the bottom surface;
an engagement member disposed within the housing, spaced apart from the rigid member in an initial position and configured to rotate about an axis; and
a cutting element coupled to the engagement member;
wherein displacement of the movable portion of the top surface moves the rigid member into contact with the engagement member and produces rotation of the engagement member and the cutting element about the axis;
wherein the cutting element rotates through the opening to form an incision in the skin then to a locked position within the housing, the locked position securing the cutting element away from the skin;
wherein the sensor and the wire advance through the opening and into the incision when the movable portion of the top surface is displaced; and
wherein the engagement member comprises a gear and the rigid member comprises a rod, the gear having teeth configured to mesh with the rod during the rotation.

9. A wearable device for continuous glucose monitoring, comprising:
a housing having a top surface opposite a bottom surface, a portion of the top surface moveable relative to the bottom surface, the bottom surface having an opening and configured to secure directly to skin of a patient;
a puck assembly disposed within the housing, the puck assembly having a sensor, a wire coupled to the sensor, and a plurality of contacts;
a rigid member disposed within the housing and configured to move relative to the bottom surface;
an engagement member disposed within the housing, spaced apart from the rigid member in an initial position and configured to rotate about an axis; and
a cutting element coupled to the engagement member;
wherein displacement of the movable portion of the top surface moves the rigid member into contact with the engagement member and produces rotation of the engagement member and the cutting element about the axis;
wherein the cutting element rotates through the opening to form an incision in the skin then to a locked position within the housing, the locked position securing the cutting element away from the skin;
wherein the sensor and the wire advance through the opening and into the incision when the movable portion of the top surface is displaced; and
wherein the engagement member comprises a disk and the rigid member comprises a plunger positioned to impart rotation to the disk.

10.-11. (canceled)

12. wearable device for continuous glucose monitoring, comprising:
a housing having a top surface opposite a bottom surface, a portion of the top surface moveable relative to the bottom surface, the bottom surface having an opening and configured to secure directly to skin of a patient;
a puck assembly disposed within the housing, the puck assembly having a sensor, a wire coupled to the sensor, and a plurality of contacts;
a rigid member disposed within the housing and configured to move relative to the bottom surface;
an engagement member disposed within the housing, spaced apart from the rigid member in an initial position and configured to rotate about an axis;
a cutting element coupled to the engagement member;
a circuit board having terminals disposed within the housing and configured to electrically couple to the plurality of contacts of the puck assembly; and
a stepped shaft extending from the top surface and a complementary recess in the circuit board, the stepped shaft being received within the recess when the top surface is actuated, the stepped shaft engaging the recess to secure the top surface in a collapsed position;
wherein displacement of the movable portion of the top surface moves the rigid member into contact with the engagement member and produces rotation of the engagement member and the cutting element about the axis;
wherein the cutting element rotates through the opening to form an incision in the skin then to a locked position within the housing, the locked position securing the cutting element away from the skin; and
wherein the sensor and the wire advance through the opening and into the incision when the movable portion of the top surface is displaced.

13.-20. (canceled)
